(19) United States
(12) Patent Application Publication
(10) Pub. No.: US 2015/0080300 A1
(43) Pub. Date: Mar. 19, 2015

(54) GROWTH FACTOR CONCENTRATE AND THE USE THEREOF

(71) Applicant: Bestop Group Holdings Limited, Hong Kong (HK)

(72) Inventors: Kin Yip Chan, Hong Kong (HK); Wing Man Tam, Hong Kong (HK)

(73) Assignee: BESTOP GROUP HOLDINGS LIMITED, Hong Kong (HK)

(21) Appl. No.: 14/487,113

(22) Filed: Sep. 16, 2014

(30) Foreign Application Priority Data
Sep. 17, 2013 (HK) 13110684.5

(51) Int. Cl. A61K 38/18; A61Q 19/08; A61K 8/64

(52) U.S. Cl. CPC A61K 38/1858; A61K 8/64; A61Q 19/08; A61K 2800/52
USPC 514/8.2; 530/399

(57) ABSTRACT

Provided herein are growth factor concentrates, cosmetic compositions and methods for cosmetic treatment. The growth factor concentrates comprise decapsulated growth factors derived from platelet rich plasma.

### CROSS REFERENCE TO RELATED APPLICATIONS

This application claims the benefit of Hong Kong Short-term Patent Application No. 13110684.5 filed on Sep. 17, 2013, the entire contents of which are incorporated herein by reference.

### FIELD OF INVENTION

This invention relates to a growth factor concentrate and the use thereof, in particular the use in cosmetic treatment.

### BACKGROUND OF INVENTION

Growth factors are a group of naturally occurring proteins or steroid hormones that are capable of stimulating cellular growth, proliferation and cellular differentiation. Growth factors are important for regulating a variety of cellular processes and play an important part in maintaining healthy skin structure and function.

Some types of cells, such as keratinocytes making up the epidermis and dermis of the skin can secret growth factors. However, the concentration of growth factor may be decreased age-relatedly. Since the skin should be fertilized by growth factors theoretically, some cosmetic products or cosmetic treatments nowadays are applying growth factors on the surface of the skin, in which the growth factors are extracted from the subject in need thereof. The cosmetic effects which are expected by applying growth factors on the skin surface include but not limited to: reducing the fine lines, expression lines and wrinkles as a result of new collagen synthesis; reducing the dark spots and pigmentation; improving the density, smoothness and firmness; and reducing the uneven skin texture and tone.

Platelet rich plasma (PRP) extracted from the subject in need thereof is used for growth factor treatment. However, the PRP must be used freshly, and cannot be stored for a long period of time. The average life span of platelets is typically only 5 to 9 days after extraction. In skin-care products, growth factors would be used repeatedly, and possibly over long periods of time. A normal cosmetic treatment of growth factors requires several times of treatments spanning a period of 3 to 6 months. Typically, every time before receiving treatment, the subject in need thereof has to suffer venipuncture and wait for at least 30 minutes for the PRP to be extracted. Some products use additives such as preservative or other chemical to extend the shelf life of growth factors.

Moreover, the absorption of the applied growth factors on the skin surface may not be as well as expected because of the block of epidermis. In addition, the PRP treatment known in the art does increase the number of platelets surrounding skin cells, but only a small amount of growth factors can be released from platelets at a slow rate.

It is necessary to provide no-additive and stable growth factors self-provided by the subject in need with a high released concentration and a longer shelf life.

### SUMMARY OF INVENTION

In the light of the foregoing background, it is an object of the present invention to provide a growth factor concentrate for cosmetic treatment to a subject in need thereof, in which the growth factor concentrate comprises decapsulated growth factors derived from platelet rich plasma.

In an exemplary embodiment of the present invention, the platelet rich plasma is extracted from the subject.

In another exemplary embodiment of the present invention, the growth factor concentrate is preserved by steps of:
a. extracting a whole blood sample from the subject, wherein the platelet rich plasma with the growth factors are contained therein;
b. obtaining the platelet rich plasma from the blood sample;
c. stabilizing the platelet rich plasma;
d. decapsulating the growth factors of the stabilized platelet rich plasma;
e. filtering out the decapsulated growth factors;
f. freeze-drying the filtered growth factors to obtain the growth factor concentrate; and
g. preserving the freeze-dried growth factor concentrate.

The growth factor concentrate is preserved for an extended period of time.

In a further exemplary embodiment of the present invention, the decapsulating step further comprises the step of freezing and thawing the platelet rich plasma. In another exemplary embodiment, the platelet rich plasma is frozen at a temperature between −50° C. and −60° C. for at least 30 minutes; in yet another exemplary embodiment, the platelet rich plasma is thawed at a temperature of 37° C. for 10 minutes. In one exemplary embodiment, the decapsulated growth factors are filtered through a cellulose acetate membrane in the filtering step. In yet another exemplary embodiment, the freezedrying step further comprises the steps of: f1. freezing the filtered growth factors at a temperature between −50° C. and −60° C. for at least 3 hours; and f2. lyophilizing the frozen growth factors at a temperature between −40° C. and −60° C. and at a pressure of about 0.18 millibar for at least 10 hours. In another exemplary embodiment, the freeze-dried growth factor concentrate is preserved at a temperature ranging from 2° C. to 10° C. in the preserving step.

In another exemplary embodiment of the present invention, the growth factor concentrate is a cryo-crystalized growth factor concentrate.

In yet another exemplary embodiment of the present invention, the growth factor concentrate has a shelf life of at least 22 months.

According to another aspect of the present invention, an anti-aging cosmetic composition for a subject in need thereof is provided, in which the anti-aging cosmetic composition comprises an effective amount of the growth factor concentrate as described above.

In another aspect of the present invention, a non-invasive method for promoting skin rejuvenation in a subject in need thereof is provided, in which the method comprises topically applying a cosmetic composition comprising an effective amount of the growth factor concentrate as described above.

In a further aspect of the present invention, a method for rejuvenating the skin of a subject in need thereof is provided, in which the method comprises the steps of:
a. cleaning the target area of the skin of the subject;
b. applying a probe from a device generating radio frequency on the cleaned skin to create channels within the cleaned skin; and
c. applying a cosmetic composition comprising an effective amount of the growth factor concentrate on the cleaned skin from step b such that the cosmetic composition can penetrate into deeper layers of the cleaned skin through the channels.

### BRIEF DESCRIPTION OF FIGURES

FIG. 1 shows the standard derivation of contamination of the extracted growth factor concentrate and fresh platelet in a study on the safety of the growth factor concentrate provided in the present invention.

FIG. 2 shows the density of released growth factors under the epidermis of the subject in need of the growth factor concentrate applied by the method provided in the present invention and fresh growth factor applied by the injection.

FIG. 3 shows the total cell number after the treatment of the growth factor concentrate provided in the present invention and a typical treatment using PRP.

FIG. 4 shows results of the studies of the application of the growth factor concentrate provided in the present invention.

### DETAILED DESCRIPTION OF THE PREFERRED EMBODIMENTS

As used herein and in the claims, “comprising” means including the following elements but not excluding others.

### Preparation of Growth Factor Concentrate

### 1. Collecting PRP

30 ml-60 ml whole blood sample was extracted from the subject by venipuncture or any other suitable means and stored in a sodium citrate blood collection tube or any other suitable containers for ease of transport.

The PRP layer was separated from blood cell layer by centrifuge at 3000 rpm or any other suitable means. The growth factors are now concentrated in the separated PRP solution.

### 2. Stabilizing PRP Layer

A buffering agent was added to the separated PRP layer to stabilize the pH value of PRP within a desired range to avoid undesired fluctuations of pH value in temperature extremities. Such undesired fluctuations of pH value may affect the efficacy of growth factors in the following steps including temperature change.

The buffering agent that could be added to the PRP solution as a lyoprotectant is Tris Buffer.

### 3. Decapsulating Growth Factor

Platelets are prone to apoptosis at low storing temperature which may release inhibitors and destroy the growth factors therein. Besides, as mentioned above, absorption rate of growth factors by the skin cell may be low, since only a small amount of growth factors are released from the platelets during the treatment. In the course of invention, the inventors intended to decapsulate the growth factors from platelets as the decapsulated growth factors tend to be less vulnerable to inhibitors and easier to be absorbed by the skin cell.

There are several methods that could release (decapsulate) growth factors from platelets, including but not limited to freezing-thawing, mechanical lysis, liquid homogenization, sonication and manual grinding. Freezing-thawing method is employed in one embodiment to decapsulate growth factors from the stabilized PRP solution.

The stabilized PRP solution was frozen at a temperature of −50° C. to −60° C. for 30 minutes, and then thawed in water bath at 37° C. for 10 minutes. The above freezing-thawing cycle was repeated for 3 more times. By way of freezing and thawing the aforesaid ice crystal formation in the platelets expands in volume and eventually ruptures the platelet membrane; thus growth factors are released from platelets and a decapsulated growth factor solution is then obtained.

By decapsulating the growth factors from platelets, the preservation period of growth factors can be much extended and the absorption rate can also be increased.

### 4. Filtrating the Decapsulated Growth Factor

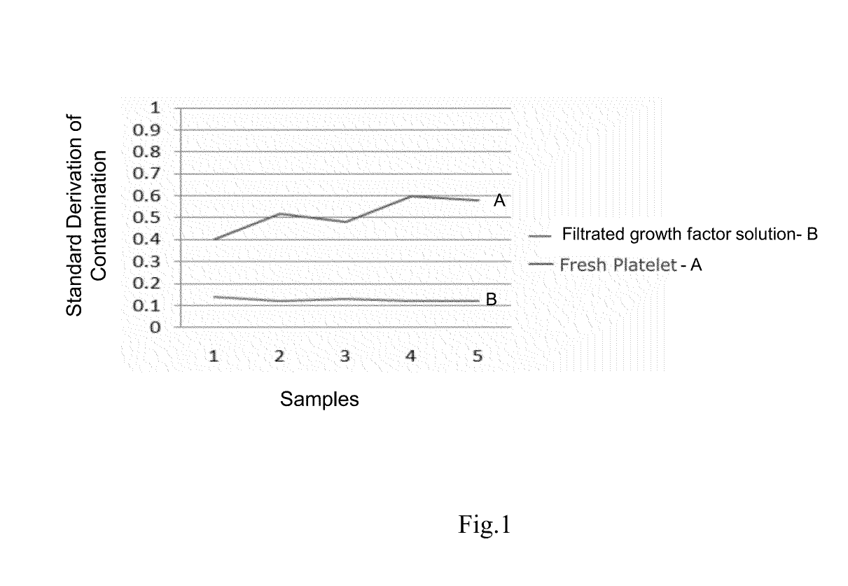

To reduce the amount of pathogens and other contaminants such as bacteria, red blood cells or cell fragments which may affect the sterility of the solution and the life span of the growth factors, the decapsulated growth factor solution was filtrated by a 0.2 μm cellulose acetate membrane. As shown in FIG. 1, filtrated growth factor solution clearly has a lower standard derivation of contamination under the same condition than that of fresh platelet, showing that filtrated growth factor solution is safer than fresh platelet.

The filtrated growth factor solution was then distributed into serum vials that were sealed with rubber stoppers and frozen. In one embodiment, these vials were frozen at a temperature between −50° C. and −60° C. for at least 3 hours to prepare for freeze-drying.

### 5. Freeze-Drying

Freeze-drying, also known as cryodesiccation, works by freezing the material and then reducing the surrounding pressure to allow the frozen water in the material to sublimate directly from the solid phase to the gas phase. This method has been used in preserving perishable material or making the material more convenient for transport.

The vials containing the filtrated growth factor solution were retrieved from storage; in one embodiment of the present invention, the storage temperature was −55° C. The loosely covered vials were then put into a freeze-dryer. In one embodiment, the freeze-drying process was maintained at a temperature between −40° C. and −60° C. and at a pressure of about 0.18 millibar, for at least ten hours until samples of growth factors were dried thoroughly. The growth factor concentrate was obtained in a form of powder (also known as cryo-crystalized growth factor, C-GF).

### 6. Storing

Upon visual inspection of the vials for sufficient dryness of the powder, the vials were resealed, sealed air-tight and removed from the freeze-dryer to a temperature, in one embodiment, of 2° C.-10° C. with humidity of lower than 70%, which can be stored up to six months. In another embodiment of the present invention, the growth factor concentrate prepared from the aforesaid steps and stored in a typical household refrigerator may then have shelf life as long as 22 months.

Administering the End Product with Growth Factor Concentrate

The growth factor concentrate prepared and preserved from the aforesaid steps could be used in the cosmetic treatment to subject in need. In one embodiment, the growth factor concentrate could be applied as a serum on areas of treatment by rehydrating with 1:1 normal saline or be administered in form of powder directly on open moist wounds or ulcers.

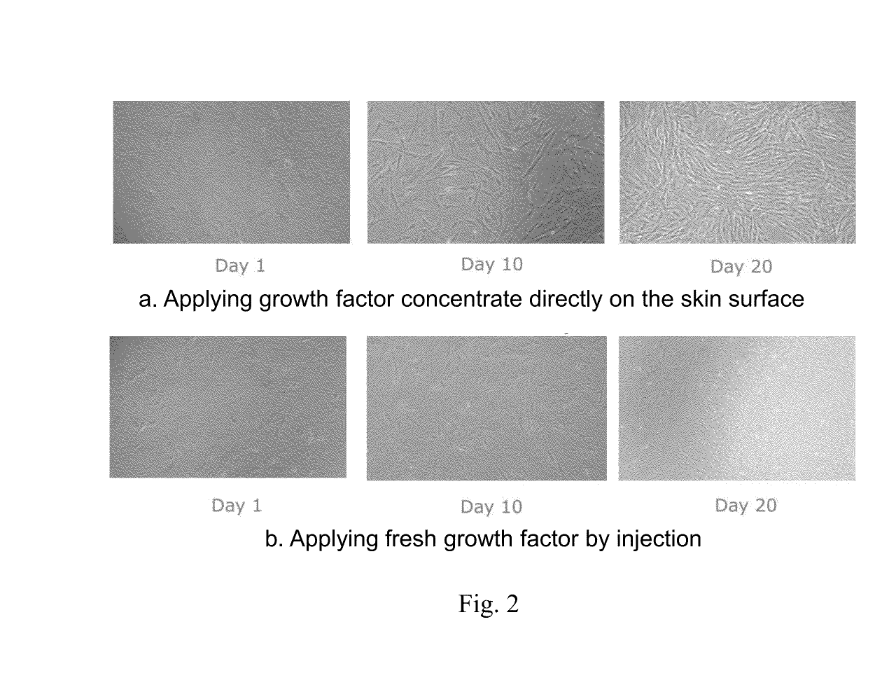

The results of the administration of the growth factor concentrate of the present invention in cosmetic treatment are shown in FIGS. 2, 3 and 4. FIG. 2 shows that there is a significant 10-time proliferation of cell number in applying the growth factor concentrate of the present invention directly on the skin surface as compared to that in applying fresh growth factor by injection. It shows that by applying the growth factor concentrate of the present invention directly on the skin surface, the be-treated skin area would have more released growth factors than that by applying fresh growth factor by injection.

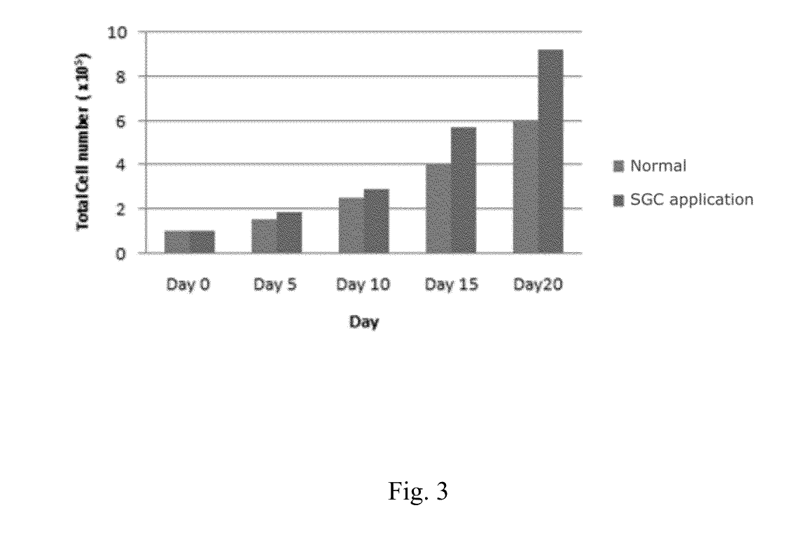

FIG. 3 reveals the results of a study on the total cell number after the treatment of traditional growth factor and the growth factor concentrate of the present invention. The cell numbers were shown to increase by using both kinds of growth factors. However, after 20 days of the treatment, the number of cell by using the growth factor concentrate of the present invention was 1.5 times of that of using traditional growth factor.

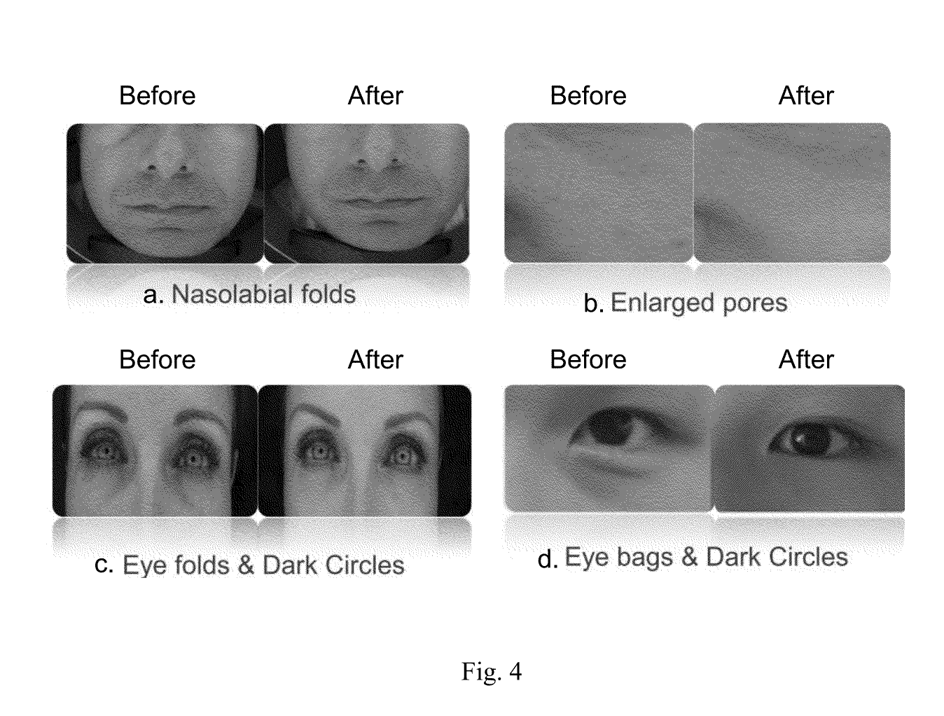

FIG. 4 shows the results of actual clinical examples before and after the treatment using the growth factor concentrate of the present invention. It shows that the growth factor concentrate of the present invention could reduce nasolabial/eye folds, enlarged pores and eye bags; reduce dark spots and pigmentation; and improve the density, smoothness and firmness.

### Applying on the Skin Surface

To improve the absorption rate of the growth factor concentrate during the treatment to the subject in need directly on the skin surface, bipolar radiofrequency (RF) technology is used in one embodiment of the present invention. Radiofrequency technology as a rejuvenation method is partially based on skin remodeling following a controlled injury and could be used for laser skin resurfacing, dermabrasion and deep chemical peels.

One embodiment of the present invention provides a method comprising steps of:
a. cleaning the target area of the skin of the subject;
b. massaging the cleaned skin with a probe of a radiofrequency machine; and
c. applying a cosmetic composition comprising an effective amount of the growth factor concentrate of the present invention to the massaged skin.

In the same embodiment, a 10 J/cm³ RF energy with 1.7 MHz is adapted. By massaging the skin before applying the growth factor concentrate, the growth factor concentrate can then easily permeate through the epidermis and the concentration thereof under the epidermis would be increased.

The exemplary embodiments of the present invention are thus fully described. Although the description referred to particular embodiments, it will be clear to one skilled in the art that the present invention may be practiced with variation of these specific details. Hence this invention should not be construed as limited to the embodiments set forth herein.

For example, the present invention is applicable on different kinds of growth factors including and not limited to Platelet Derived Growth Factor (PDGF), Transforming Growth Factor Beta (TGF-B), Insulin-like Growth Factor (IGF-1), Platelet Factor-4 (PF-4), Vascular Endothelial Growth Factor (VEGF), Epidermal Growth Factor (EGF), Hepatocyte Growth Factor (HGF), Bone Morphogenetic Proteins (BMPs) and Fibroblast Growth Factor (FGF).

What is claimed is:

1. A growth factor concentrate for cosmetic treatment to a subject in need thereof, wherein the growth factor concentrate comprises decapsulated growth factors derived from platelet rich plasma.

2. The growth factor concentrate of claim 1, wherein the platelet rich plasma is extracted from the subject.

3. The growth factor concentrate of claim 1, wherein the growth factor concentrate is preserved for an extended period of time by steps of:
extracting a whole blood sample from the subject, wherein the platelet rich plasma with the growth factors are contained therein;
obtaining the platelet rich plasma from the blood sample;
stabilizing the platelet rich plasma;
decapsulating the growth factors from the platelets of the stabilized platelet rich plasma;
filtering out the decapsulated growth factors;
freeze-drying the filtered growth factors; and
preserving the freeze-dried growth factor concentrate.

4. The growth factor concentrate of claim 3, wherein the decapsulating step comprises the step of freezing and thawing the platelet rich plasma.

5. The growth factor concentrate of claim 4, wherein in the freezing step, the platelet rich plasma is frozen at a temperature between −50° C. and −60° C. for 30 minutes.

6. The growth factor concentrate of claim 4, wherein in the thawing step, the platelet rich plasma is thawed at a temperature of 37° C. for 10 minutes.

7. The growth factor concentrate of claim 3, wherein in the filtering step, the decapsulated growth factors are filtered through a cellulose acetate membrane.

8. The growth factor concentrate of claim 3, wherein the freeze-drying step comprises the steps of:
freezing the filtered growth factors at a temperature between −50° C. to −60° C. for at least 3 hours; and
lyophilizing the frozen growth factors at a temperature between −40° C. and −60° C. and at a pressure of about 0.18 millibar for at least 10 hours.

9. The growth factor concentrate of claim 3, wherein in the preserving step, the freeze-dried growth factors is preserved at a temperature ranging from 2° C. to 10° C.

10. The growth factor concentrate of claim 1, wherein in the growth factor concentrate is a cryo-crystalized growth factor concentrate.

11. The growth factor concentrate of claim 1, wherein in the growth factor concentrate has a shelf life of at least 22-month.

12. An anti-aging cosmetic composition for a subject in need thereof, comprising an effective amount of the growth factor concentrate of claim 1.

13. A non-invasive method for promoting skin rejuvenation in a subject in need thereof, comprising topically applying a cosmetic composition comprising an effective amount of the growth factor concentrate of claim 1.

14. A method for rejuvenating the skin of a subject in need thereof, comprising the steps of:
cleaning the target area of the skin of the subject;
massaging the cleaned skin with a probe of a radiofrequency machine; and
applying a cosmetic composition comprising an effective amount of the growth factor concentrate of claim 1 to the massaged skin.
